(19) United States
(12) Patent Application Publication
(10) Pub. No.: US 2020/0308458 A1
(43) Pub. Date: Oct. 1, 2020

(54) ADHESIVE COMPOSITIONS COMPRISING IONIC COMPOUNDS

(71) Applicant: NITTO DENKO CORPORATION, Osaka (JP)

(72) Inventors: Yufen Hu, San Diego, CA (US); Hongxi Zhang, Temecula, CA (US); Peng Wang, San Diego, CA (US)

(21) Appl. No.: 16/765,713

(22) PCT Filed: Nov. 20, 2018
(86) PCT No.: PCT/US2018/061996

§ 371 Date: May 20, 2020

Related U.S. Application Data
(60) Provisional application No. 62/589,460, filed on Nov. 21, 2017.

(51) Int. Cl. C09J 11/06; C09J 133/08; C07C 211/63; C07D 233/64; C09J 9/02; C08K 5/19; C08K 3/32; C08K 5/3445

(52) U.S. Cl. CPC C09J 11/06; C09J 133/08; C07C 211/63; C07D 233/64; C09J 2433/00; C08K 5/19; C08K 3/32; C08K 5/3445; C09J 9/02

(57) ABSTRACT

The present disclosure generally relates to ionic compositions which may be used in or as an adhesive material for selectively adhering two items together. More particularly, but not exclusively, the present disclosure relates to ionic compositions that include a mixture of ammonium and imidazolium cations with an anionic sulfonylimide compound or a mixture of various imidazolium cations with an anionic sulfonylimide compound.

### CROSS-REFERENCE TO RELATED APPLICATIONS

This application claims the benefit of U.S. Provisional Patent Application No. 62/589,460 filed Nov. 21, 2017, the content of which is incorporated herein by reference in its entirety.

### BACKGROUND

The present disclosure generally relates to ionic compounds and compositions including the same which may be used in or as an adhesive material or coating for selectively adhering two items together. More particularly, but not exclusively, the present disclosure relates to compositions that include first and second ionic compounds, and which may be applied to an underlying substrate and then released therefrom upon the application of an electric potential. In one aspect, a composition includes a mixture of ammonium and imidazolium cationic compounds with an anionic sulfonylimide compound or a mixture of various imidazolium cationic compounds with an anionic sulfonylimide compound.

Certain compositions are known which may be used as an adhesive coating that is applied to an electrically conductive surface of a first substrate. The adhesive coating may be sandwiched between the electrically conductive surface of the first substrate and an electrically conductive surface of a second substrate in order to adhere or join the first and second substrates together. Upon the application of an electric potential, the adhesive coating is de-bonded from one or both of the first and second substrates in order to separate the first and second substrates from one another. It has been observed however that certain forms of this type of coating may have an undesired corrosive effect on the electrically conductive surfaces to which they are applied. Thus, there remains a need for further contributions in this area of technology.

The subject matter disclosed and claimed herein is not limited to embodiments that solve any disadvantages or that operate only in environments such as those described above. Rather, this background is only provided to illustrate examples of where the present disclosure may be utilized.

### SUMMARY

The present disclosure generally relates to ionic compounds and compositions including the same which may be used in or as an adhesive material or coating for selectively adhering two items together. More particularly, but not exclusively, the present disclosure relates to compositions that include first and second ionic compounds, and which may be applied to an underlying substrate and then released therefrom upon the application of an electric potential. In one aspect, a composition includes a mixture of ammonium and imidazolium cationic compounds with an anionic sulfonylimide compound or a mixture of various imidazolium cationic compounds with an anionic sulfonylimide compound.

In one embodiment, a composition includes a first cationic compound having the following structure:

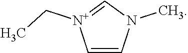

The composition also includes a second cationic compound according to Formula (I) or

Formula (II), where Formula (I) has the following structure

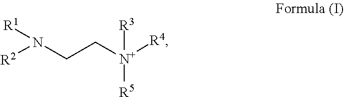

wherein each of R¹, R², R³, R⁴ and R⁵ independently represents a C₁-C₃ alkyl, a C₁-C₃ alkoxy, or a C₁-C₃ alkoxy, and where Formula II has the following structure

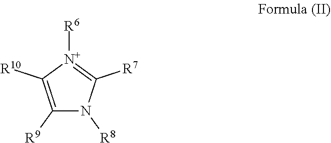

wherein R⁶ represents a C₁-C₃ alkyl or an optionally substituted C₃-C₁₂ alkylamine, each of R⁷, R⁹, and R¹⁰ independently represents hydrogen or a C₁-C₃ alkyl, and R⁸ represents a C₁-C₃ alkyl or an optionally substituted C₃-C₁₂ alkylamine, provided that R⁶ does not represent ethyl if R⁸ represents methyl.

In another embodiment, an adhesive composition includes a mixture of a first ionic compound and a second ionic compound. The first ionic compound exhibits a first degree of corrosiveness with respect to a metallic material which is greater than a corresponding second degree of corrosiveness exhibited by the second ionic compound with respect to the metallic material. In addition, the mixture of the first ionic compound and the second ionic compound exhibits a corresponding third degree of corrosiveness with respect to the metallic material which is less than the first degree of corrosiveness. When applied to the metallic material, the composition including the mixture of the first ionic compound and the second ionic compound may be selectively released from the metallic material upon the application of an electric potential.

In another embodiment, a method involves adhering a first substrate to a second substrate with a composition according to one of the foregoing embodiments.

Additional features and advantages will be set forth in the description which follows, and in part will be obvious from the description, or may be learned by practice. The features and advantages may be realized and obtained by means of the instruments and combinations particularly pointed out in the appended claims. These and other features will become more fully apparent from the following description and appended claims.

### BRIEF DESCRIPTION OF THE DRAWINGS

FIG. 1 is a schematic illustration of the use of an ionic composition described herein for adhering two substrates together.

FIG. 2 is a schematic illustration of the release or de-bonding of the two substrates of FIG. 1 upon application of an electric potential.

FIG. 3 is a schematic illustration of an apparatus used for testing adhesion properties of an ionic composition described herein.

FIG. 4 is a graphical illustration of peeling strength density vs. time of the ionic composition tested in connection with FIG. 3.

FIG. 5 is a pictorial representation of the testing of the corrosive effect of various ionic compositions described herein.

### DETAILED DESCRIPTION

For purposes of promoting an understanding of the present disclosure, reference will now be made to the following embodiments and specific language will be used to describe the same. It will nevertheless be understood that no limitation of the scope of the disclosure is thereby intended, such alterations and further modifications in the described subject matter, and such further applications of the disclosed principles as described herein being contemplated as would normally occur to one skilled in the art to which the disclosure relates.

The present disclosure generally relates to ionic compounds and compositions including the same which may be used in or as an adhesive material or coating for selectively adhering two items together. More particularly, but not exclusively, the present disclosure relates to compositions that include first and second ionic compounds, and which may be applied to an underlying substrate and then released therefrom upon the application of an electric potential. In one aspect, a composition includes a mixture of ammonium and imidazolium cations with an anionic sulfonylimide compound or a mixture of various imidazolium cations with an anionic sulfonylimide compound.

As used herein, when a compound or chemical structural is referred to as being “optionally substituted” it includes a feature that has no substituents (i.e. unsubstituted), or a feature that is “substituted,” meaning that the feature has one or more substituents. A substituted group is derived from the unsubstituted parent structure wherein one or more hydrogen atoms on the parent structure have been independently replaced by one or more substituent groups. A substituent group may have one or more substituent groups on the parent group structure. In one or more forms, the substituent groups may be independently selected from an optionally substituted alkyl or alkenyl, —O-alkyl or alkoxy (e.g. —OCH₃, —OC₂H₅, —OC₃H₇, —OC₄H₉, etc.), —S-alkyl or alkylsulfone (e.g., —SCH₃, —SC₂H₅, —SC₃H₇, —SC₄H₉, etc.), —NR′R″, —OH, —SH, —CN, —NO₂, or a halogen, wherein R′ and R″ are independently hydrogen or an optionally substituted alkyl. Wherever a substituent is described as “optionally substituted,” that substituent can be substituted with the above substituents.

As used herein, the term “amino” refers to the overall uncharged or net uncharged chemical group having the following structure:

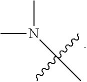 [SMILES: *N(C)C]

As used herein, the term “ammonium” refers to the overall charged or net charged chemical compound: NR⁴⁺.

As used herein, the term “imidazolium” refers to the overall charged or uncharged ring system having the following structure:

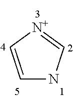

As used herein, the term “bis(fluorosulfonyl)imide” and/or “sulfonyl imide” refers to a heteroatom moiety having, for example, the following structure:

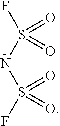

In one embodiment, an ionic composition includes a first cationic imidazolium compound having the following structure:

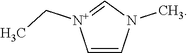

The ionic composition also includes a second cationic compound. In one form, the second cationic compound is a basic cationic compound. For example, the second cationic compound may be an ammonium cation which has the following general structure:

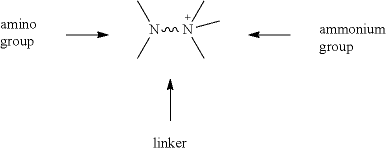

In one form, the cationic compound of this nature includes at least one aliphatic amine which may have two substituent groups. Additionally or alternatively, the at least one aliphatic amine may include an amino group. Still, in another form, the cationic compound of this nature includes a second aliphatic amine which may have three substituent groups. In one aspect of this form, the second aliphatic amine includes an ammonium group. In one or more forms, the linker in the cationic compound of this nature is a C₀-C₅ aliphatic chain such as, for example, a methyl, ethyl or propyl group.

In one more particular form, the second cationic compound is a compound according to Formula (I):

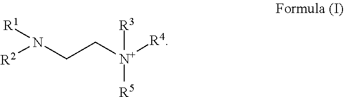

In one form, each of R¹, R², R³, R⁴ and R⁵ independently represents a C₁-C₃ alkyl, a C₁-C₃ alkoxy, or a C₁-C₃ alkoxy. In another form, each of R¹, R², R³, R⁴ and R⁵ independently represents a C₁-C₃ alkyl. It should be appreciated that, in some forms, one or more of R¹, R², R³, R⁴ and R⁵ may represent or include a hydrophobic functional group. When present, the hydrophobic functional group can include an optionally substituted alkyl group such as a methyl, ethyl and/or a propyl group.

In one particular form, the second cationic compound is one of the following compounds or a combination or mixture thereof:

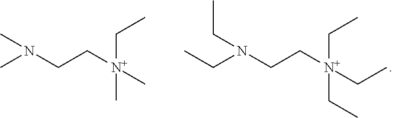

As another example, the second cationic compound may be an imidazolium cationic compound which has the following general structure:

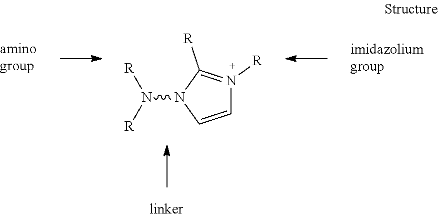

In one form, the cationic compound of this nature includes at least one amine which may be an aliphatic amine that may also optionally include two substituent groups. Additionally or alternatively, the at least one aliphatic amine may include an amino group. Still, in another form, the cationic compound of this nature includes a second amine which may be an aryl amine that may also optionally include two substituent groups. In one particular form, the aryl amine includes an imidazolium group. In one or more forms, the linker in the cationic compound of this nature may be a C₀-C₅ aliphatic chain such as, for example, methyl, ethyl, or propyl group.

In one more particular form, the second cationic compound is an imidazolium cationic compound according to Formula (II):

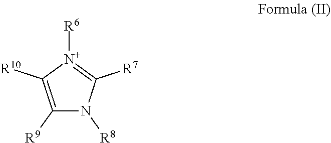

With respect to any relevant structural representation, such as Formula II, in some embodiments, R⁶ represents a C₁-C₃ alkyl or an optionally substituted C₃-C₁₂ alkylamine. In some embodiments, R⁶ represents a C₁-C₃ alkyl or a C₂ alkylamine. In some embodiments, R⁶ represents a C₁-C₅ alkyl. In some embodiments, R⁶ represents a C₂ alkylamine. In some embodiments, R⁶ represents a C₁ alkyl. In some embodiments, R⁶ represents a C₂ alkyl. In some embodiments, R⁶ represents a 1-(2-(diisopropylamino)ethyl).

With respect to any relevant structural representation, such as Formula II, in some embodiments, R⁷ represents a C₁-C₃ alkyl or H. In some embodiments, R⁷ represents hydrogen. In some embodiments, R⁷ represents C₁-C₅ alkyl. In some embodiments, R⁷ represents a substituted C₂ alkyl.

With respect to any relevant structural representation, such as Formula II, in some embodiments, R⁸ represents a C₁-C₃ alkyl or an optionally substituted C₃-C₁₂ alkylamine. In some embodiments, R⁸ represents a C₂ alkylamine. In some embodiments, R⁸ represents a 1-(2-(diisopropylamino)ethyl. In some embodiments, R⁸ represents a C₁ alkyl.

With respect to any relevant structural representation, such as Formula II, in some embodiments, R⁹ represents hydrogen or a C₁-C₃ alkyl. In some embodiments, R⁹ represents hydrogen.

With respect to any relevant structural representation, such as Formula II, R¹⁰ represents hydrogen or a C₁-C₃ alkyl. In some embodiments, R¹⁰ represents hydrogen.

In one form, R⁶ represents a C₁-C₃ alkyl or an optionally substituted C₃-C₁₂ alkylamine, each of R⁷, R⁹, and R¹⁰ independently represents hydrogen or a C₁-C₃ alkyl, and R⁸ represents a C₁-C₃ alkyl or an optionally substituted C₃-C₁₂ alkylamine. In one aspect of this form, R⁶ does not represent ethyl if R⁸ represents methyl. In another more particular form, R⁶ represents a C₁-C₃ alkyl or a C₂ alkylamine, R⁷ represents a C₁-C₃ alkyl or H, R⁸ represents a C₂ alkylamine, and R⁹ and R¹⁰ represent hydrogen. In still another particular form, each of R⁷, R⁹, and R¹⁰ represents hydrogen, R⁶ represents a C₁-C₅ alkyl, and R⁸ represents a C₂ alkylamine. In yet another form, each of R⁹ and R¹⁰ represents hydrogen, R⁷ represents C₁-C₅ alkyl, and each of R⁶ and R⁸ represents a C₂ alkylamine. In still another form, R⁶ represents a C₁ alkyl, R⁷ represents hydrogen, R⁸ represents a 1-(2-(diisopropylamino)ethyl), and each of R⁹ and R¹⁰ represents hydrogen. In yet another form, R⁶ represents a 1-(2-(diisopropylamino)ethyl), R⁷ represents a substituted C₂ alkyl, R⁸ represents a 1-(2-(diisopropylamino)ethyl), and each of R⁹ and R¹⁰ represents a hydrogen. Forms in which R⁶ represents a C₂ alkyl, each of R⁷, R⁹ and R¹⁰ represents hydrogen, and R⁸ represents a C₁ alkyl are also contemplated.

It should be appreciated that, in some forms, one or more of R⁶, R⁷, R⁸, R⁹ and R¹⁰ may represent or include a hydrophilic functional group. When present, the hydrophilic functional group may include nitrogen, sulfur and/or phosphorous. In one particular form, the hydrophilic functional group includes an amino group.

In one particular form, the second cationic compound, when an imidazolium cationic compound, is one of the following compounds or a combination or mixture thereof:

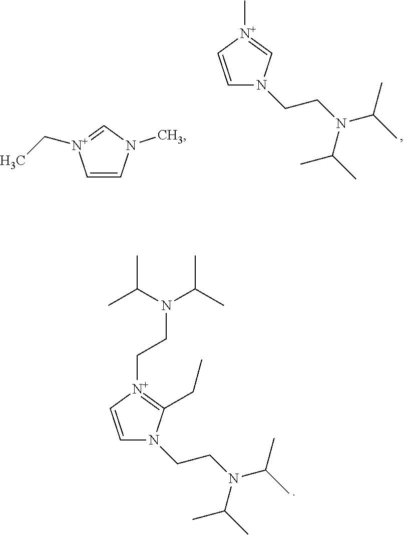

The ionic composition also includes one or more anionic compounds. For example, the first and second cationic compounds may be part of ionic compounds which include the same or different anionic compounds. In one form, the one or more anionic compounds are a sulfonylsulfonic amide anion(s). In one particular form, the sulfonylsulfonic amide anion includes a fluoroalkysulfonylamide compound. In another particular form, the fluorosulfonylamide compound has the following structure:

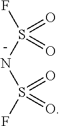

While not previously discussed, it should be appreciated that the ionic compositions described herein may exhibit reduced Lewis acidity which may, for example, result in reduced corrosiveness to metallic materials to which they may be applied. In some aspects, the ionic composition can include a suitable pH. In some aspects, the ionic composition can include a pH that is not overly acidic or overly basic. In some examples, the pH can range from about 5 to about 9, or about 6 to about 8 or about 7. When alkaline, the pH can range from about 7 to about 9, about 7.5 to about 8.5, or about 8. In addition, it is contemplated that the ionic compositions disclosed herein may have a generally reduced size such as less than 160 g/mole. While also not previously discussed, it should be appreciated that the ionic compositions disclosed herein may include a mixture of the first and second cationic compounds that includes about 95% of the first cationic compound and about 5% of the second cationic compound. Alternatively, the mixture may include about 5% of the first cationic compound and about 95% of the second cationic compound. In another form, the mixture may include about 40-60% of the first cationic compound and 40-60% of the second cationic compound. In yet another form, the mixture may include about 50% of the first cationic compound and about 50% of the second cationic compound. However, other variations are also contemplated.

The ionic compositions described herein may be utilized as, or in, an adhesive material which may be used to bond two or more items together in a releasable fashion; i.e., it may provide a selectively debondable layer. Stated alternatively, the adhesive material may be used to selectively bond the items together, allowing for the adhesive material to be de-bonded from one or more of the items and facilitate separation of the items if desired. More particularly, an adhesive material according to the present disclosure may be provided on an electrically conductive surface of a first substrate, and an electrically conductive surface of a second substrate may be positioned in contact with the adhesive material in order to bond or join the first and second substrates together. In this configuration, the adhesive material is sandwiched between the first and second substrates, although other variations are contemplated. As indicated above, if desired, the adhesive material facilitates de-bonding and separation of the first and second substrates. More specifically, upon the application of an electric potential, the adhesive material will be de-bonded or released from the conductive surface of one or both of the substrates, resulting in separation of the first and second substrates from one another.

The selectively debondable layer can be used in a selectively debondable structure to adhere two non-conductive materials to one another, and then release the bonding so that the debonded materials do not contain any conductive materials or layers. This type of structure comprises an electro-conductive layer with a selectively debondable layer adhered to each side. Each of these adhesive layers can then be adhered to a nonconductive material, thus providing adhesion between two nonconductive structures. An electromotive force can then be applied to the electro-conductive layer to reduce the adhesion in both adhesive layers. Thus, the two nonconductive structures can be adhered to one another, and then separated, without needing to first be bonded or attached to a conductive layer or material.

While not previously described, it should be appreciated that the compositions disclosed herein may include components in addition to the cationic and anionic compounds. For example, in one form, the compositions may also include a polymer. Non-limiting examples of polymers which could be present in the compositions described herein include those described in WO2017/064918 and/or JP2017-075289, which are incorporated herein by specific reference in their entirety. In one form, the polymer may have a glass transition temperature below 0° C., although other variations are possible. In some aspects, the polymer can be an acrylic polymer. In some aspects, the acrylic polymer can include a monomer unit derived from a monomer of a formula R^{a}CH═CHCO₂R^{b}, wherein R^{a} is H or C₁₋₁₄ alkyl (e.g. methyl, ethyl, C₃ alkyl, C₄ alkyl, C₅ alkyl, C₆ alkyl, etc.), and R^{a} is H or C₁₋₁₄ alkyl (e.g. methyl, ethyl, C₃ alkyl, C₄ alkyl, C₅ alkyl, C₆ alkyl, etc.). In some embodiments, the polymer includes repeating units derived from acrylic acid, methyl acrylate, methacrylic acid, methylmethacrylate, or a combination thereof. In some aspects, the acrylic polymer can contain an alkyl-methacrylate ester and a monomer unit derived from a monomer that contains a polar group. In one form, the acrylic polymer may be an acrylate polymer, an alkylacrylate polymer, an alkyl-alkylacrylate ester polymer, or a combination thereof. In some aspects, the polymer comprises an acrylate polymer, a methacrylate polymer, or a combination of both acrylate and methacrylate polymers. In one aspect, an acrylic polymer contains a monomer unit derived from a C₁-C₁₄ alkyl group containing alkyl (meth)acrylate ester. In other forms however, the acrylic polymer can contain a monomer unit derived from a C₁-C₁₄ alkyl or alkoxy group. In one form, the acrylic polymer may contain an alkyl (meth)acrylate ester, and a monomer unit derived from a polar group containing monomer. In one aspect of this form, the polar group containing monomer may be a carboxyl group containing monomer. In an additional or alternative aspect of this form, the C₁-C₁₄ alkyl group containing alkyl (meth)acrylate ester is butyl (meth)acrylate. In some aspects, the C₁₋₁₄ alkyl group containing alkyl (meth)acrylate ester is butyl-methacrylate ester, and may be methyl-methacrylate ester, ethyl-methacrylate ester, propyl-methacrylate ester, methyl-ethylacrylate ester, methyl-propylacrylate ester, methyl-butylacrylate ester, or other alkyl-alkylacrylate ester.

While not previously discussed, it should be appreciated that the polymer may be crosslinked. The crosslinked polymer may include the polymer crosslinked with only polymers in the composition. In some aspects, the crosslinked polymer may chemically crosslink with an ammonium cation. In some aspects, the crosslinked polymer may chemically crosslink with the fluorosulfonylimide anion. In some aspects, the crosslinked polymer may chemically crosslink with the ammonium cation and fluorosulfonylimide anion. Crosslinkers that can crosslink the polymers can be selected based on the desired properties in order to provide the crosslinked polymer. The crosslinkers may be suitable for use with the alkyl-alkylacrylate esters. In one form for example, the polymer is crosslinked with an epoxy crosslinker such as is N,N,N′,N′-tetraglycidyl-m-xylenediamine, just to provide one non-limiting example. However, it should be recognized that any suitable crosslinker may be used to crosslink the polymer. The crosslinker can be selected to retain the selective adhesive properties and selective debonding properties as described herein. The crosslinker can also be selected to retain the anticorrosive properties described herein.

Any suitable amount of ionic liquid described herein, alone or in combination, may be used in the adhesive composition. In some embodiments, the ionic liquid or ionic compound is about 0.0-1%, about 1-2%, about 2-3%, about 3-4%, about 4-5%, about 5-6%, about 6-7%, about 7-8%, about 8-9%, about 9-10%, about 10-15%, about 15-20%, about 20-25%, about 25-30%, about 30-40%, about 40-50, about 50-100%, about 4.5-5.0%, or about 5% of the total weight of the ionic liquid plus the polymer.

It is contemplated that the compositions described herein could be utilized for a number of different applications, including for example in a device as disclosed in JP2017-075289 and/or WO2017/064925, which are incorporated herein by specific reference in their entirety. Accordingly, the device can be an electronic device that includes an electro-conductive substrate having the selectively adhesive compositions described herein. In some aspects, the device can include a battery.

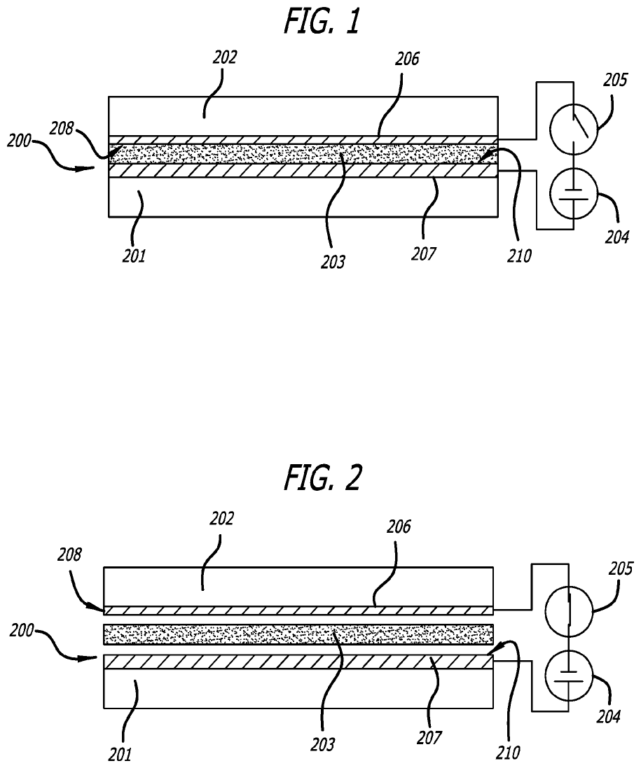

Referring now to FIGS. 1 and 2, additional details regarding the use of an ionic composition described herein for selectively bonding two substrates together in apparatus 200 will be provided. An adhesive material 203 which includes an ionic composition described herein provides a layer or coating positioned between electrically conductive surface 206 of substrate 202 and electrically conductive surface 207 of substrate 201. In some embodiments, adhesive material 203 can include the adhesive composition of one of the embodiments formed into an adhesive layer; and at least one release liner on at least one side of the adhesive layer. In some aspects, the adhesive member can include a release liner on each side of the adhesive layer. The release liner may be removed to expose a side of the adhesive layer so that the adhesive layer can be adhered to another surface.

In one form, one or both of substrates 201, 202 may be formed of an electrically conductive material such that one or both of electrically conductive surfaces 206, 207 is/are formed of the same material as the remainder of substrates 201, 202. However, it is possible in other forms to use one or more electrically conductive materials for electrically conductive surfaces 206, 207 which are different from the material(s) forming substrates 201, 202. Similarly, it should be appreciated that one or both of substrates 201, 202 could be formed of one or more materials which are not electrically conductive, such as wood, cardboard, fiberglass density fiberboard or polymeric/plastic materials, provided that surfaces 206, 207 or some portion thereof are electrically conductive. In some aspects, substrates 201 and 202 can be electrical insulators. In some aspects, substrates 201 and 202 may be semiconductors. Any of the non-electro-conductive substrates 201 or 202 or semiconductor substrate (e.g., printed circuit board, PCB) can have any thickness and may be coupled to other substrates, materials or devices. In these forms, electrically conductive surfaces 206, 207 may be provided as a coating or layer on substrates 201, 202.

In the illustrated form, electrically conductive surfaces 206, 207 are electrically coupled to or in electrical communication with a power source 204 in a closeable electrical circuit that includes an intervening switch 205. In one form, power source 204 may be a direct current power supply that provides a DC voltage in the range of about 3V to 100V, although other variations are contemplated. When switch 205 is closed, the electrical potential is applied between electrically conductive surfaces 206, 207 in order to de-bond adhesive material 203 from one or both of electrically conductive surfaces 206, 207 and, as a result, allow substrates 201 and 202 to be physically separated from one another. For example, while not intending to be bound by any particular theory, it is believed that a movement of ions within adhesive material 203 may be effected by application of the electrical potential thereto. Upon a sufficient amount of movement being effected, e.g., sufficient ionic components appear adjacent to the electro-conductive surface, the adhesive qualities of the adhesive material is reduced, enabling separation of electro-conductive surfaces 206, 207 and/or adhesive material 203.

In one form, one or both of substrates 201, 202 may include an electrically conductive carbonaeceous material or an electrically conductive metal. As suggested above, one or both of substrates 201, 202 may also include an electrically conductive layer which may be formed of a metallic material such as, but not limited to, aluminum. The electrically conductive layer may include a conventional material such as a metal, mixed metal, alloy, metal oxide, and/or composite metal oxide, or it may include a conductive polymer. Examples of suitable metals for the electrically conductive layer include the Group 1 metals, the metals in Groups 4, 5, and 6, and the Group 8-10 transition metals. Further examples of suitable metals for the electrically conductive layer include stainless steel, Al, Ag, Mg, Ca, Cu, Mg/Ag, LiF/Al, CsF, and/or CsF/Al and/or alloys thereof. In some embodiments, the electro-conductive layers (e.g., first electro-conductive surface 206 and second electro-conductive surface 207) and/or the adhesive layer can each have a thickness in the range of about 1 nm to about 1000 μm, or 1 nm to about 100 μm, or 1 nm to about 10 μm, or 1 nm to about 1 μm, or 1 nm to about 0.1 μm, or 10 nm to about 1000 μm, or 100 nm to about 1000 μm, or 1 μm to about 1000 μm, or 10 μm to about 1000 μm, or 100 μm to about 1000 μm. In some aspects, the thickness can be from 20 nm to about 200 μm, or 100 nm to about 100 μm, or 200 nm to about 500 μm.

While not previously discussed, it should be appreciated that the ionic compositions described herein may provide various properties which are desirable for certain applications. For example, in some forms, the ionic compositions disclosed herein may eliminate or reduce corrosion of the electrically conductive surfaces on which they are positioned. In one form for example, the ionic compositions disclosed herein include components which reduce the acidity of the environment immediately adjacent to the electrically conductive surfaces. In one aspect, an adhesive material may include one or more materials, in addition to the cationic and anionic compounds themselves, which may be used to reduce the corrosiveness of the ionic cations and/or anions immediately adjacent the electrically conductive surfaces. The corrosive effect of an adhesive material may be assessed pursuant to the procedures described in ASTM G69-12 (Standard Test Method for Measurement of Corrosion Potentials of Aluminum Alloys). Additional procedures for assessing the corrosive effect of an adhesive material on the electrically conductive surfaces are described in the Examples of the subject application. For example, one suitable alternative protocol to assess the corrosive effect of the ionic composition upon the electro-conductive surface or material(s) can be achieved by visually examining the interface between the ionic composition (or the material in which the ionic composition is included) and the electro-conductive surface or material(s) (e.g., aluminum foil) for any indication of corrosive degradation of the substrate and/or dissolution of the material from the electro-conductive substrate (e.g., metal) into the ionic composition (or the material in which the ionic composition is included) and/or pitting of the surface of the electro-conductive substrate.

In one form, an adhesive material including an ionic composition disclosed herein may be chemically stable relative to an electrically conductive electrode or an electrically conductive material; i.e., there is a lack of (or minimal presence of) undesired reactions between a metal material/electrode and the adhesive material. Undesired reactions may include, for example, corrosive degradation of the metal material/electrode, dissolution of the metal in the selectively adherent adhesive and/or pitting of the metal material/electrode. An adhesive material including an ionic composition disclosed herein may be chemically stable relative to aluminum, stainless steel and/or mixtures thereof, just to provide a few examples. In one form, contact of an adhesive material including an ionic composition disclosed herein upon an electrically conductive surface may result in the absence of, or minimize, any corrosive degradation of the surface for a period of at least or greater than 15 minutes, 30 minutes, 1 hour, 3 hours, 5 hours, 7 hours, 24 hours, 50 hours, 100 hours, 125 hours, 200 hours, 300 hours and/or 400 hours. In some forms, direct contact of an adhesive material including an ionic composition disclosed herein upon an electrically conductive surface may minimize and/or prevent corrosive degradation of the surface for one of the time periods identified above in an environment of 60° C. and 90% relative humidity (RH), 85° C. and 85% RH, or 90° C. and 80% RH. In one form, the absence of any corrosive degradation can be demonstrated by a lack of total penetration of an electrically conductive 50 nm thick sheet of aluminum foil for one of the time periods identified above and/or at the environmental conditions identified above.

In one form, an adhesive material including an ionic composition described herein may be formulated to minimize corrosion of an electrically conductive surface under conditions of prolonged high humidity and high temperature. For example, an adhesive composition may be capable of maintaining two substrates in fixed relation to each other during and after being subjected to Accelerated Aging Test Method II (preferably after exposure to 85° C. and 85% RH for one of the periods of time identified above).

### EXAMPLES

It should be appreciated that the following Examples are for illustration purposes and are not intended to be construed as limiting the subject matter disclosed in this document to only the embodiments disclosed in these examples.

### Example 1: Synthesis of 2-(Dimethylamino)-N-ethyl-N,N-dimethylethan-1-aminium bis(fluorosulfonyl)amide

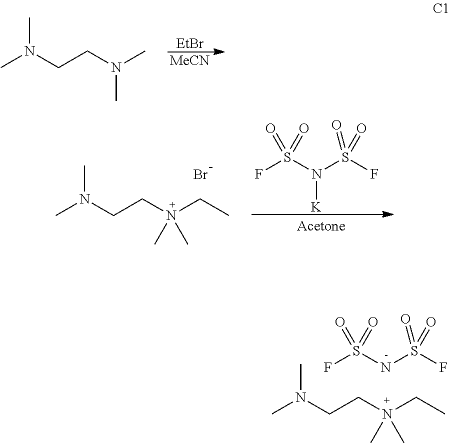

A solution of N,N,N′,N′-tetramethyl-1,2-ethylenediamine (15 mL, 100 mmol) in dry acetonitrile (75 mL) was placed in a pressure reactor. Ethyl bromide (3.7 mL, 50 mmol) was added, and the reactor was sealed and heated at 60° C. for 16 h. The volatiles were removed under reduced pressure. Trituration of the residue with ethyl ether (200 mL) produced a dense precipitate. The solid was filtered off, washed with ethyl ether and dried in a vacuum oven to give 2-(dimethylamino)-N-ethyl-N,N-dimethylethan-1-aminium bromide (10.58 g, 94% yield).

A mixture of 2-(dimethylamino)-N-ethyl-N,N-dimethylethan-1-aminium bromide (8.02 g, 35.6 mmol), KFSI (7.80 g, 35.6 mmol) and dry acetone (100 mL) was stirred under argon at 60° C. for 2 h. After cooling to room temperature, the solid was filtered off, and the solvent was removed under reduced pressure to give an oily substance. A solution of the crude product in ethyl acetate (200 mL) was washed with water (100 mL), dried over sodium sulfate and concentrated under reduced pressure to give pure 2-(dimethylamino)-N-ethyl-N,N-dimethylethan-1-aminium bis(fluorosulfonyl)amide (9.60 g, 88% yield). ¹H NMR (d₆-DMSO) δ: 3.38 (q, J=7.2 Hz, 2H), 3.35 (t, J=6.2 Hz, 2H), 3.03 (s, 6H), 2.62 (t, J=6.2 Hz, 2H), 2.20 (s, 6H), 1.24 (t, J=7.2 Hz, 3H).

### Example 2: Synthesis of 2-(Diethylamino)-N,N,N-triethylethan-1-aminium bis(fluorosulfonyl)amide

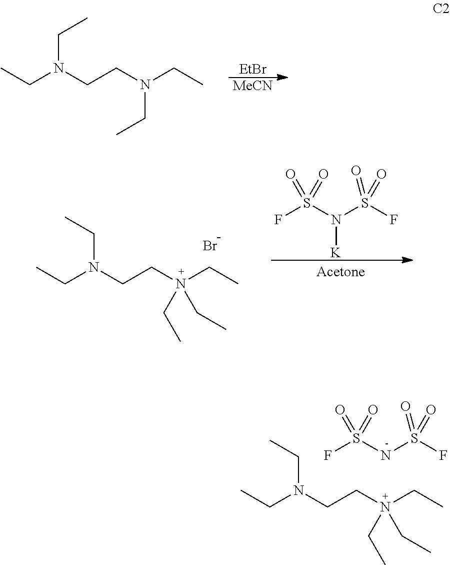

A solution of N,N,N′,N′-tetraethylethane-1,2-diamine (10.1 g, 58.6 mmol) in dry acetonitrile (65 mL) was placed in a pressure reactor. Ethyl bromide (4.79 g, 43.96 mmol) was added, and the reactor was sealed and heated at 60° C. for 16 h. The volatiles were removed under reduced pressure. Trituration of the residue with ethyl ether (75 mL) resulted in the slow recrystallization of the crude product. The white crystals were filtered off, washed with ethyl ether (100 mL) and dried in a vacuum oven for 2 hours at room temperature to give 2-(diethylamino)-N,N,N-triethylethan-1-aminium bromide (11.22 g, 68% yield).

A mixture of 2-(diethylamino)-N,N,N-triethylethan-1-aminium bromide (5.61 g, 19.9 mmol), KFSI (4.37 g, 19.9 mmol) and dry acetone (100 mL) was stirred under argon at 50° C. for 2 h. After cooling to room temperature, the solid was filtered off, and the solvent was removed under reduced pressure to give a crude product. Dichloromethane (75 mL) was added to the crude product and the resulting mixture sat overnight. The fine white precipitates were filtered and the filtrate was concentrated under reduced pressure to give pure 2-(diethylamino)-N,N,N-triethylethan-1-aminium bis(fluorosulfonyl)amide (7.14 g, 94% yield). ¹H NMR (400 MHz, DMSO-d₆) δ 3.29 (q, J=7.2 Hz, 6H), 3.23 (t, J=6.9 Hz, 2H), 2.68 (t, J=6.8 Hz, 2H), 2.58-2.44 (m, 4H), 1.19 (t, 9H), 0.98 (t, J=7.1 Hz, 6H).

### Example 3: Synthesis of 1-(2-(diisopropylamino)ethyl)-3-methyl-1H-imidazol-3-ium bis(fluorosulfonyl)amide

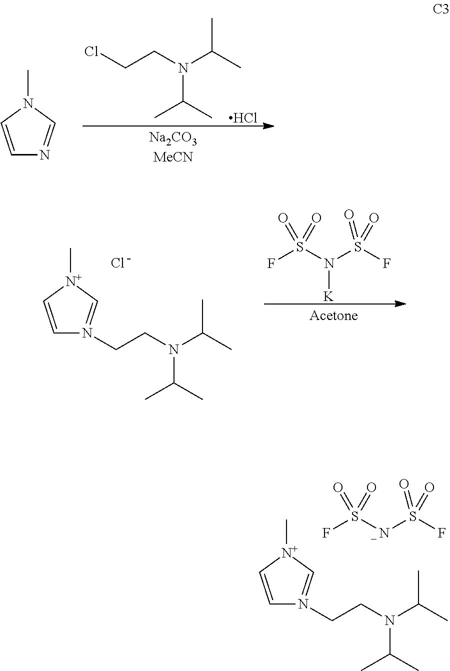

1-methyl-1H-imidazole (3.99 g, 48.6 mmol), 2-diisopropylaminoethyl chloride hydrochloride (10.21 g, 51.0 mmol), and sodium carbonate (14 g, 132 mmol) in dry acetonitrile (80 mL) were placed in a round-bottomed flask. The reaction mixture was refluxed under Argon for 24 h. After cooling to room temperature, the reaction mixture was filtered through Celite and the filtrate was concentrated under reduced pressure to obtain a crude product. Trituration of the residue with ethyl ether (100 mL) was performed. The white solid was filtered off, washed with ethyl ether (2×50 mL) and dried in a vacuum oven for 3 hours at 50° C. to give 1-(2-(diisopropylamino)ethyl)-3-methyl-1H-imidazol-3-ium chloride (10.36 g, 87% yield).

A mixture of 1-(2-(diisopropylamino)ethyl)-3-methyl-1H-imidazol-3-ium chloride (5.0 g, 20.3 mmol), KFSI (4.46 g, 20.3 mmol) and dry acetone (100 mL) was stirred under argon at 50° C. for 2 h. After cooling to room temperature, the solid was filtered off, and the solvent was removed under reduced pressure to give a crude product. Dichloromethane (100 mL) was added to the crude product and the resulting mixture sat overnight. The fine white solid was filtered and the filtrate was concentrated under reduced pressure to give pure 1-(2-(diisopropylamino)ethyl)-3-methyl-1H-imidazol-3-ium bis(fluorosulfonyl)amide (7.64 g, 96% yield). ¹H NMR (400 MHz, DMSO-d₆) δ 9.03-8.97 (m, 1H), 7.73 (t, J=1.8 Hz, 1H), 7.67 (t, J=1.8 Hz, 1H), 4.10 (t, J=5.8 Hz, 2H), 3.87 (s, 3H), 2.96 (hept, J=6.6 Hz, 2H), 2.73 (t, 2H), 0.85 (d, J=6.6 Hz, 12H).

### Example 4: Synthesis of 1,3-bis(2-(diisopropylamino)ethyl)-2-ethyl-1H-imidazol-3-ium bis(fluorosulfonyl)amide

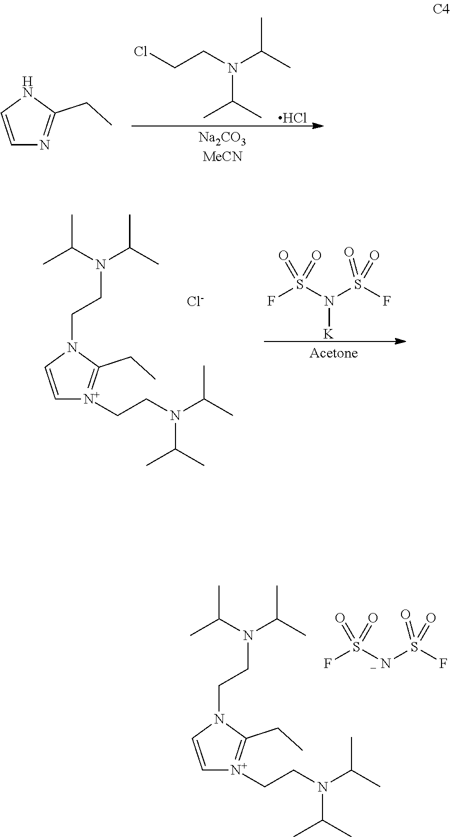

2-ethyl-1H-imidazole (4.67 g, 48.6 mmol), 2-diisopropylaminoethyl chloride hydrochloride (10.21 g, 51.0 mmol), and sodium carbonate (14 g, 132 mmol) in dry acetonitrile (80 mL) were placed in a round-bottomed flask. The reaction mixture was refluxed under Argon for 24 h. After cooling to room temperature, the reaction mixture was filtered through celite and the filtrate was concentrated under reduced pressure to obtain a crude product. Trituration of the residue with ethyl ether (100 mL) was performed. The white solid were filtered off, washed with ethyl ether (2×50 mL), and further purified with recrystallization in MeCN/ethyl ether until the mono-substituted product was no longer present. The purified product was dried in a vacuum oven for 3 hours at 50° C. to give 1,3-bis(2-(diisopropylamino)ethyl)-2-ethyl-1H-imidazol-3-ium chloride (3.35 g, 18% yield).

A mixture of 1,3-bis(2-(diisopropylamino)ethyl)-2-ethyl-1H-imidazol-3-ium chloride (3.35 g, 8.65 mmol), KFSI (1.897 g, 8.65 mmol) and dry acetone (80 mL) was stirred under argon at 50° C. for 2 h. After cooling to room temperature, the solid was filtered off, and the solvent was removed under reduced pressure to give a crude product. Dichloromethane (100 mL) was added to the crude product and the resulting mixture sat overnight. The fine white solid was filtered and the filtrate was concentrated under reduced pressure to give pure 1,3-bis(2-(diisopropylamino)ethyl)-2-ethyl-1H-imidazol-3-ium bis(fluorosulfonyl)amide (4.42 g, 96% yield). ¹H NMR (400 MHz, DMSO-d₆) δ 7.70 (s, 2H), 4.09 (t, J=5.9 Hz, 4H), 3.09 (q, J=7.6 Hz, 2H), 3.00 (hept, J=6.6 Hz, 4H), 2.76 (t, J=5.9 Hz, 4H), 1.26 (t, J=7.6 Hz, 3H), 0.88 (d, J=6.6 Hz, 24H).

### Example 5: Synthesis of 1-ethyl-3-methyl-imidazolium bis(fluorosulfonyl)amide (C5)

1-ethyl-3-methyl-imidazolium bis(fluorosulfonyl)amide can be made as described in U.S. Pat. No. 7,901,812.

### Example 6: Preparation of Polymer Solution

95 mass parts n-butyl acrylate, 5 mass parts acrylic acid and 125 mass parts ethyl acetate were introduced into a stirring flask attached to a condenser that was equipped with a nitrogen gas inlet. The mixture was stirred at room temperature, while introducing the nitrogen gas, for about 1 hour to remove oxygen from the reaction system. 0.2 mass parts azobisisobutyronitrile (AIBN) were added, which increased the temperature of the resulting mixture to about 63° C.±2° C., and the resulting mixture was mixed/stirred for about 5-6 hours for polymerization. After stopping the reaction, an acrylic polymer-containing solution having a solid content of about 30% resulted. The apparent molecular weight of the polymer solution (P1) was determined to be about 800,000, with a glass transition temperature (Tg) of about −50° C.

### Example 7: Preparation of Adhesive Sheets

Adhesive sheets were prepared by mixing the polymer solution described above in Example 6 with 0.01 g of an epoxy crosslinking agent, such as N,N,N′,N′-tetraglycidyl-m-xylenediamine, per 100 g of solid polymer solution, and an ionic liquid including either compound C5, a combination of compounds C5 and C1, a combination of compounds C5 and C2, a combination of compounds C5 and C3, or a combination of compounds C5 and C4, to obtain electrically debondable adhesive compositions. The prepared compositions were coated/deposited upon a surface treated PET separator (release liner) [MRF38, made by Mitsubishi Chemical Corp., Japan], forming an adhesive composite layer at a thickness of about 150 microns (μm). The coated film was then heat dried at 130° C. for about 3 minutes. A second PET separator (release liner) was then aligned over the exposed adhesive coating to obtain a layered sheet (PET separator/adhesive coating/PET separator) which was then aged/dried at 50° C. for about 20-24 hours and then stored under ambient conditions until needed.

### Example 8: Adhesive Ionic Composition Corrosive Test

Just prior to the application of the adhesive sheets described in Example 7 to a nano-Al coating layer on a PET film, the aforementioned release liner was removed from each adhesive sheet. The adhesive sheets were then applied to the metallic surface of the film (50 nm-thick aluminum coated PET film [Toray Advanced Film, Tokyo, Japan]).

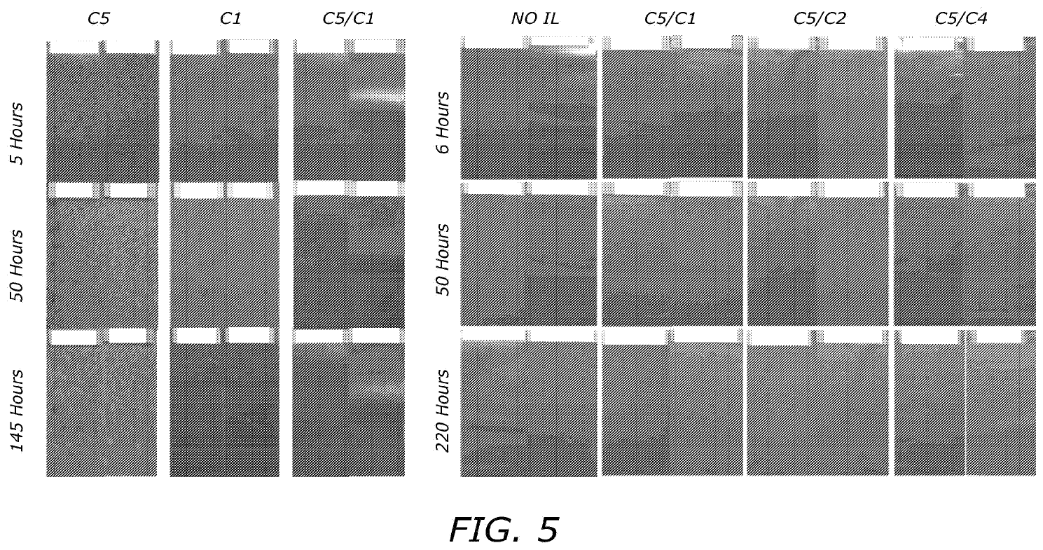

The prepared films were placed in a Temperature & Humidity Benchtop chamber, set at 60° C./85% Relative Humidity (RH), 85° C./85% RH or 80° C./90% RH (ESPEC North America, [Hudsonville, Mich., USA], Criterion Temperature & Humidity Benchtop Model BTL-433) and were periodically checked at selected times (initially hourly). As shown in FIG. 5, the interface between the adhesive and the aluminum foil was visually examined for an indication of corrosive degradation of the aluminum foil, dissolution of the metal in the selectively adherent adhesive, and/or pitting of the aluminum foil. If corrosiveness was observed, the time was recorded and the sample was indicated as corrosive. The results are shown in Table 1 below.

TABLE 1

| No Ionic | IL including | IL including | IL including | IL including | IL including |
|---|---|---|---|---|---|
| Liquid | compound | compounds | compounds | compounds | compounds |
| (IL) | C5= | C1 & C5 | C2 & C5 | C3 & C5 | C4 & C5 |
| >480 h | <6 h | <480 h | >480 h | <480 h | >480 h |

### Example 9: Electrical De-bonding of Adhesive Composition Testing

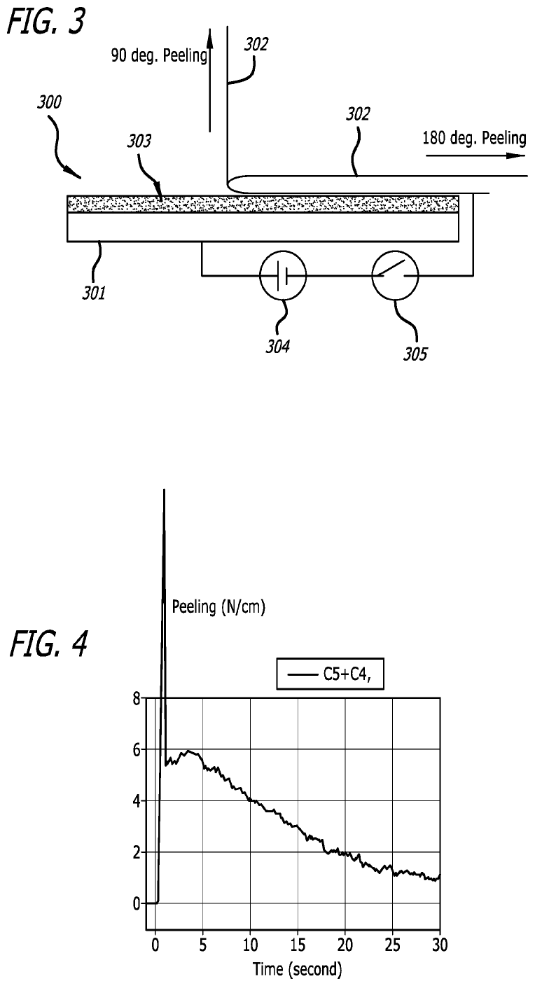

Testing for electrical de-bonding or release of an adhesive composition was done in the manner as described in JP2017-095590 and/or WO2017/064918, and also shown in apparatus 300 of FIG. 3. As shown in FIG. 3, adhesive material 303 (including a mixture of compounds C4 and C5) was coated upon a conductive substrate 301 which was 25 mm wide and 100 mm long. The resulting substrate 301 was laminated upon another flexible conductive layer 302 (such as aluminum foil and/or metalized plastic film such as PET), which was 10 mm to 25 mm wide and 100 mm longer than substrate 301. The lamination was conducted by the application of rolling pressure by a 2 kg roller and roll press.

The bonding/de-bonding tester (Mark-10, Copiague, N.Y., USA, model ESM303 motorized tension/compression stand) was equipped with a Mark-10 force gauge (Series 7-1000) and had lower and upper clamps. The conductive substrate 301 was fixed onto the lower clamp and then electrically connected to the positive pole of a power supply 304 (Protek DC Power Supply 3006B). Flexible conductive layer 302 was fixed to the upper clamp which was connected with the negative pole of the same DC power supply. The power supply had an output range from 0 to 100 VDC. The moving/peeling speed was set at 300 mm/min A switch 305 is present and, when closed, the electrical potential is applied between substrate 301 and layer 302.

In a dynamic test, the voltage is applied a few seconds after the peeling or separation starts and the time and peeling strength readings from the force gauge are recorded by the data acquisition system (Mark-10 MESURgauge Plus). FIG. 4 shows the 180 degree peeling strength evolution with time when 10 VDC was applied to the adhesive material that is doped with a composition including compounds C4 and C5 at a concentration of 5 wt. %.

In a static de-bonding test, the sample was fixed on to the tester and connected to the power supply in the same way. The initial 180-degree peeling was measured at the same peeling speed. Then peeling was stopped. A DC voltage (10 VDC for example) was applied for some time (10 second for example). The peeling strength was then measured at the same peeling speed of 300 mm/min. For the same adhesive sample including a composition having a mixture of compounds C4 and C5, the initial peeling strength is 6.0 N/cm, and the residual adhesion peeling strength is ˜1 after applying 10 VDC for 10 second.

In one embodiment, an ionic composition is provided that includes a first ionic compound having a first corrosiveness level and a first debonding ability and a second ionic compound having a second corrosiveness level and a second debonding ability. In one form, the first corrosiveness level is higher than the second corrosiveness level, and the first debonding ability is greater than the second debonding ability. However, the ionic composition exhibits a corrosiveness level that is less than that of the first corrosiveness level and a debonding ability that is substantially the same as the first debonding ability.

In another embodiment, an ionic composition includes a first ionic compound and a second ionic compound where the cationic compound in the first ionic compound is different from the cationic compound in the second ionic compound. In addition, when applied to an electro-conductive surface, the composition may be used to bond two items together which may be selectively released from one another upon the application of an electric potential.

For the processes and/or methods disclosed, the functions performed in the processes and methods may be implemented in differing order, as may be indicated by context. Furthermore, the outlined steps and operations are only provided as examples, and some of the steps and operations may be optional, combined into fewer steps and operations, or expanded into additional steps and operations.

This disclosure may sometimes illustrate different components contained within, or connected with, different other components. Such depicted architectures are merely exemplary, and many other architectures can be implemented which achieve the same or similar functionality.

The terms used in this disclosure, and in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.). In addition, if a specific number of elements is introduced, this may be interpreted to mean at least the recited number, as may be indicated by context (e.g., the bare recitation of “two recitations,” without other modifiers, means at least two recitations, or two or more recitations). As used in this disclosure, any disjunctive word and/or phrase presenting two or more alternative terms should be understood to contemplate the possibilities of including one of the terms, either of the terms, or both terms. For example, the phrase “A or B” will be understood to include the possibilities of “A” or “B” or “A and B.”

The terms and words used are not limited to the bibliographical meanings, but, are merely used to enable a clear and consistent understanding of the disclosure. It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to those skilled in the art, may occur in amounts that do not preclude the effect the characteristic was intended to provide.

Aspects of the present disclosure may be embodied in other forms without departing from its spirit or essential characteristics. The described aspects are to be considered in all respects illustrative and not restrictive. The claimed subject matter is indicated by the appended claims rather than by the foregoing description. All changes which come within the meaning and range of equivalency of the claims are to be embraced within their scope.

1. A composition, comprising:
a first cationic compound having the following structure: 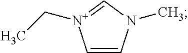
and
a second cationic compound according to Formula (I) or Formula (II), wherein Formula (I) has the following structure 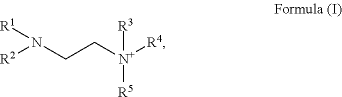
wherein each of R¹, R², R³, R⁴ and R⁵ independently represents a C₁-C₃ alkyl, a C₁-C₃ alkoxy, or a C₁-C₃ alkoxy, and wherein Formula II has the following structure 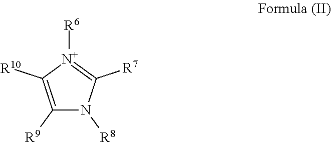
wherein R⁶ represents a C₁-C₃ alkyl or an optionally substituted C₃-C₁₂ alkylamine, each of R⁷, R⁹, and R¹⁰ independently represents hydrogen or a C₁-C₃ alkyl, and R⁸ represents a C₁-C₃ alkyl or an optionally substituted C₃-C₁₂ alkylamine, provided that R⁶ does not represent ethyl if R⁸ represents methyl.

2. The composition of claim 1, further comprising an anionic compound having the following structure:

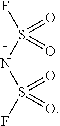

3.-4. (canceled)

5. The composition of claim 1, wherein the second cationic compound is

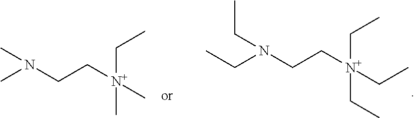

6.-8. (canceled)

9. The composition of claim 1, wherein the second cationic compound is a compound according to Formula (II) having one of the following structures:

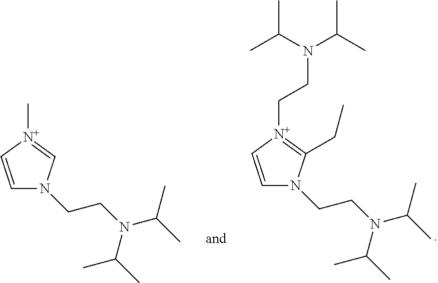 [SMILES: CC(C)N(CCn1cc[n+](C)c1)C(C)C ; CCc1n(CCN(C(C)C)C(C)C)cc[n+]1CCN(C(C)C)C(C)C]

10. The composition of claim 1, wherein the first cationic compound is part of a first ionic compound further comprising an anionic compound, and wherein the second cationic compound is part of a second ionic compound further comprising an anionic compound, wherein the anionic compound has the following structure:

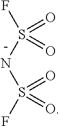

11. (canceled)

12. An apparatus, comprising a first substrate, a second substrate, and a composition according to claim 1 positioned between the first substrate and the second substrate, wherein the first substrate and the second substrate are adhered together by the composite.

13. The apparatus of claim 12, wherein the first substrate includes a first electrically conductive surface, the second substrate includes a second electrically conductive surface, and the composition is positioned in contact with the electrically conductive surfaces.

14. The apparatus of claim 13, wherein the electrically conductive surfaces comprise an electro-conductive metal, a mixed metal, an alloy, a metal oxide, a mixed metal oxide, a plastic, a carbonaceous material, a composite metal, or a conductive polymer.

15. The apparatus of claim 14, wherein at least one of the electrically conductive surfaces includes aluminum.

16. The apparatus of claim 13, further comprising a DC power supply, wherein at least one of the first electrically conductive surface and the second electrically conductive surface is in electrical communication with the DC power supply, creating a closeable electrical circuit, wherein the DC power supply is about 3 volts to about 100 volts.

17. (canceled)

18. The apparatus of claim 16, wherein the application of an electrical potential to at least one of the electrically conductive surfaces reduces the adhesion of the composition.

19. The apparatus of claim 16, wherein the first electrically conductive surface is a surface of a first electrically conductive layer and the second electrically conductive surface is a surface of a second electrically conductive layer, wherein the first electrically conductive layer and the second electrically conductive layer are about 20 nm to about 200 μm thick.

20. (canceled)

21. The apparatus of claim 16, wherein the electrically conductive surfaces are disposed upon a substrate, and wherein the substrate comprises wood, cardboard, fiberglass or non-electro-conductive plastic.

22. (canceled)

23. The apparatus of claim 16, wherein the composition has a reduced corrosive effect upon the first electrically conductive surface or the second electrically conductive surface, and wherein the reduced corrosive effect is observable under conditions of high humidity and high temperature over a period of about 15 minutes to about 300 hours.

24. (canceled)

25. A method, comprising adhering a first substrate to a second substrate with a composition according to claim 10.

26. The method of claim 25, further comprising applying an electrical potential between the first and second substrates and separating the first substrate from the second substrate.

27. (canceled)

28. An adhesive composition, comprising a mixture of a first ionic compound and a second ionic compound, wherein:
the first ionic compound exhibits a first degree of corrosiveness with respect to a metallic material which is greater than a corresponding second degree of corrosiveness exhibited by the second ionic compound with respect to the metallic material;
the mixture of the first ionic compound and the second ionic compound exhibits a corresponding third degree of corrosiveness with respect to the metallic material which is less than the first degree of corrosiveness; and
when applied to the metallic material, the composition including the mixture of the first ionic compound and the second ionic compound may be selectively released from the metallic material upon the application of an electric potential.

29. The adhesive composition of claim 28, wherein release of the composition including the mixture of the first ionic compound and the second ionic compound from the metallic material upon the application of an electric potential is achieved substantially the same as release of a corresponding second composition including the first ionic compound which exhibits a greater releasability from the metallic material than a third corresponding composition including only the second ionic compound.

30. The adhesive composition of claim 28, wherein the first ionic compound includes a first cationic compound having the following structure: 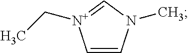
wherein the second ionic compound includes a second cationic compound according to Formula (I) having one of the following structures: 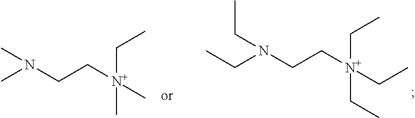
and
wherein each of the first and second ionic compounds includes an anionic compound having the following structure:

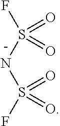

31.-40. (canceled)

41. The adhesive composition of claim 28, wherein the first ionic compound includes a first cationic compound having the following structure: 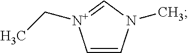
wherein the second ionic compound includes a second cationic compound according to Formula (II) having one of the following structures: 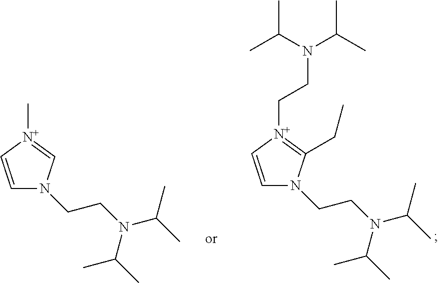 [SMILES: CC(C)N(CCn1cc[n+](C)c1)C(C)C ; CCc1n(CCN(C(C)C)C(C)C)cc[n+]1CCN(C(C)C)C(C)C]
and
wherein each of the first and second ionic compounds includes an anionic compound having the following structure: [chemical structure]
